(19) United States
(12) Patent Application Publication
(10) Pub. No.: US 2006/0259042 A1
(43) Pub. Date: Nov. 16, 2006

(54) Device for multi-ligation of hemorrhoids

(76) Inventor: Ali Mohamed Ali Hassanien, Roshdy, Alecxandria (EG)

Correspondence Address: WELSH & KATZ, LTD, 120 S RIVERSIDE PLAZA, 22ND FLOOR, CHICAGO, IL, 60606, US

(21) Appl. No.: 11/231,120

(22) Filed: Sep. 20, 2005

Related U.S. Application Data
(63) Continuation-in-part of application No. PCT/EG04/00013, filed on Apr. 28, 2004.

(30) Foreign Application Priority Data
Oct. 28, 2003 (EG) 2003101012

(51) Int. Cl. A61B 17/10

(52) U.S. Cl. 606/139

(57) ABSTRACT

The Device for ligation of veins of the hemorrhoid comprises a front part including a fixed pipe surrounded by a movable pipe, a triggering cylinder with a triggering handle of elongated cylinder construction connected between the rear of a third part and the front part, and the third part being a rear part or mechanical handle part comprising a cylinder and handle for holding the device and a mechanism including the handle for effecting suction of veins into the front part of the device by pulling the handle with a connected piston backward.

### CROSS REFERENCE TO RELATED APPLICATIONS

This application is a continuation in part of PCT application no. PCT/EG25004/000013, filed on Apr. 28, 2004 which claims priority from Egyptian application no. 2003101012 filed Oct. 28, 2003 entitled Device for Ligation of Hemorroids.

### BACKGROUND OF THE INVENTION

1. Field of the Invention

Hemorrhoid, or piles, is a painful mass of swollen veins in the lining of the anus and rectum, resulting from the formation of varicose veins around the anus. Hemorrhoids can occur at any age, often without apparent cause. Causes may include constipation, pregnancy, liver disorders, rectal tumors, and the repeated use of laxatives. Hemorrhoids can cause itching and burning and may bleed during a bowel movement.

Hemorrhoids do not cause cancer nor do they become cancerous. But any rectal bleeding may be a sign of cancer of the colon or rectum and should be checked by a physician.

Hemorrhoids is formed in this way: if bile or phlegm be determined to the veins in the rectum, it heats the blood in the veins; and these veins becoming heated attract blood from the nearest veins, and being gorged the inside of the gut swells outwardly, and the heads of the veins are raised up, and being at the same time bruised by the faeces passing out, and injured by the blood collected in them, they squirt out blood, most frequently along with the faeces, but sometimes without faeces.

Internal hemorrhoids occur above the dentate line and are, therefore, usually painless. They can cause bleeding or they may push out of the anal opening (prolapse) when they become quite large.

External hemorrhoids occur below the dentate line and are generally painful. They are sometimes called skin tags, or can be seen or felt as extra skin around the anus. External hemorrhoids can become very distressing when the blood in the external hemorrhoid clots and inflammation irritates the nearby, pain-sensitive skin. This condition is called a thrombosed external hemorrhoid and is quite painful.

2. Description of the Related Art

Rubber band ligation is an office procedure in which a small rubber band is placed at the base of the internal hemorrhoid with a special applicator. The rubber band cuts off the blood supply to the hemorrhoid and the hemorrhoid falls off in about four to five days. There is generally minimal or no discomfort associated with this procedure.

U.S. Pat. No. 3,760,810 to Van Hoorn discloses an endoscope-equipped instrument comprising a device with two tubes mounted one inside the other, with the inner tube protruding at the front of the outer tube. Means are included to move the outer tube forwardly relative to the inner tube and cause an elastic cord to be dislodged and placed about the tissue to be ligated.

In U.S. Pat. No. 4,257,419, there is disclosed an instrument for ligating hemorrhoids, wherein a suction tube fitted inside a proctoscope provides means for sucking the hemorrhoid into a suction cavity where a ligating ring is applied. Both of these instruments are rigid devices suited for treating lesions close to the external regions, and both are equipped with only a single elastic ring for treating a single lesion.

In U.S. Pat. Nos. 4,257,420 and 4,471,766, there is disclosed an instruments which equipped with a single elastic band and utilize forceps to position the tissue for ring ligation.

In U.S. Pat. No. 3,870,048, there is disclosed a ring applicator device having forceps slidably mounted in a cylinder for grasping the fallopian tube and including means for displacing an elastic ring to effect a ligation of tissue. While this device can be equipped with a plurality of elastic rings, its rigidity precludes its use with a flexible endoscope for treating the deeper regions of an internal organ, such as the alimentary tract.

In U.S. Pat. Nos. 6,149,659 and 6,685713, there is disclosed a flexible endoscopic instrument is provided with a plurality of elastic ligating bands mounted on a barrel of an endoscopic ligator affixed in coaxial relation to the insertion end of an endoscope. The bands are adapted to be dislodged therefrom in sequence at selectively controlled times for treating multiple lesions during a single insertion of the endoscope into a body organ. While this device can be equipped with a plurality of elastic bands, its mode of action depends mainly on pulling a trip wire.

In our WO05/039421 a device for multiple ligation of hemorrhoid was basically described which is incorporated herein.

### BRIEF SUMMARY OF THE INVENTION

The present invention relates to an endoscopic device for ligation of hemorrhoids as basically described in WO05/039421 characterized in that it has an internal built in mechanism for effecting an automatic suction without using any external suction.

Device is loaded with several rubber bands that can be used in one operation. The number of the rubber bands may vary from 3-6 rubber band. In another embodiment of the invention, rubber bands can be loaded as a disposable cartridge which is loaded on use.

### BRIEF DESCRIPTION OF THE SEVERAL VIEWS OF THE DRAWINGS

FIG. 1 is schematic drawings of the device according to the present invention.

FIG. 2 schematic cross section of the third part (mechanical and handle part) of the device according to the invention.

FIG. 3 schematic cross section of an alternative embodiment of the suction mechanism according to the present invention.

FIG. 4 is a schematic drawings showing embodiment of the built in suction mechanism.

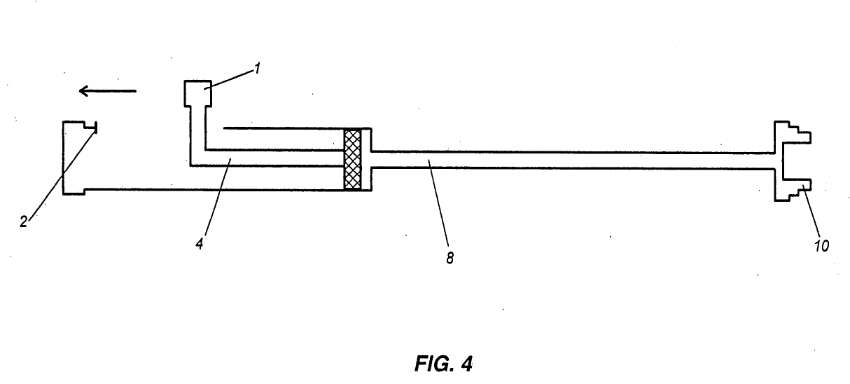

FIG. 5 is schematic drawings showing an alternative embodiment of the built in suction mechanism.

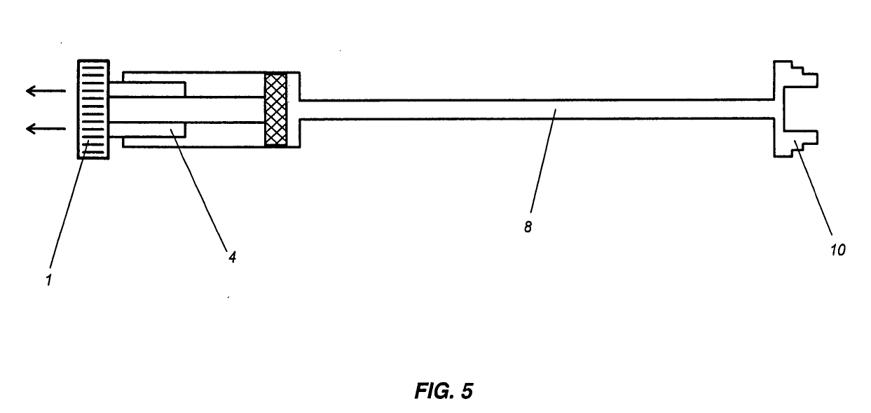

### DETAILED DESCRIPTION OF THE INVENTION

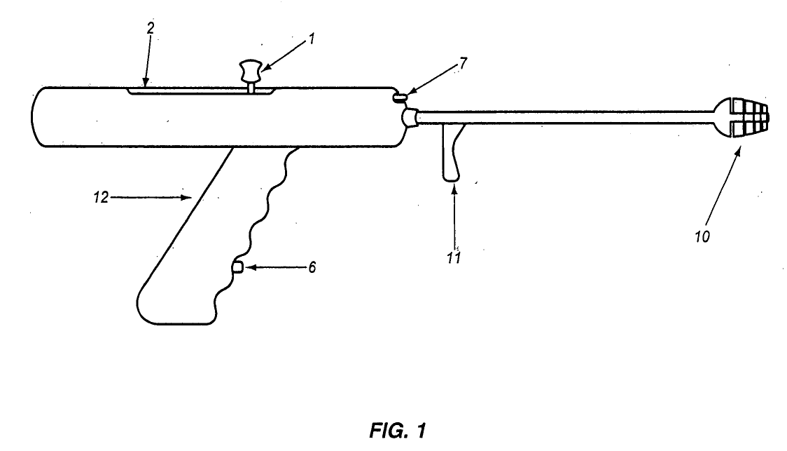

The invention can be better understood be referring to the attached drawings. FIG. 1 represents the device of the invention which has a general appearance of a pistol. The said device consists of three major parts. The first is the front part 10, which is responsible for the suction of veins of the hemorrhoid inside the device and release of the rubber band to surround it detailed construction of the said front has been described in detail in our WO 2005/039421.

The second part of the device consists of triggering cylinder 8 having triggering handle 11 which can move forward and backward within a limited space to effect triggering action and release of the rubber band.

The main third part which represent the new feature of the invention is the rear part or mechanical & handle part. This part consists of cylinder 14 and handle 12 for holding the device. The cylinder 14 has a top handle 1 which can be pulled back through the channel 2. In the front part of The cylinder 14 there is a lamp 7 that focus the light in the direction of the front part 10 to give the surgeon more concentrated lights on the working area. Handle 12 are used to hold the device and direct the front part towards the working area. There is a small button 6 which activate the lighting lamp on pressing.

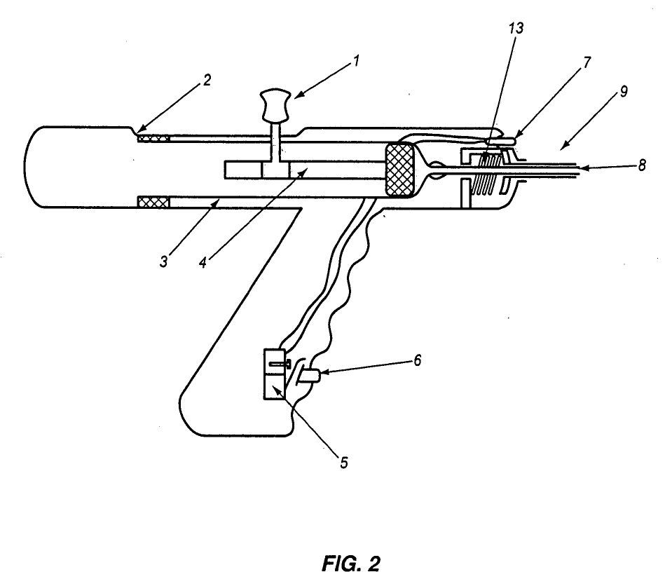

Details of the third part are better seen in FIG. 2 which represent cross section of the third part. This part consists of major cylinder 14, having inside syringe 3 with a piston 4. The said piston is connected with handle 1 on top of the cylinder 14 and can be pulled back through the channel 2 up to the end of the channel whereas it is locked in L turn extension of the channel. By pulling back the handle 1 a suction action is created through the connecting pipe 8 which connect the third part to the front of the device and thereby pulling the vein inside the front of the device whereas pressing the handle 11 result it release of the rubber band to surround the vein.

In the front part of The cylinder 14 there is a lamp 7 which is connected through electrical cables to Power source 5 ( e.g. battery), lighting circuit is activated by button 6.

The front part of the cylinder 14 incasing the mechanical part responsible for releasing the rubber band. There is an outside movable pipe 9 surrounding the fixed connecting pipe 8 the movable pipe are controlled by spring 13.

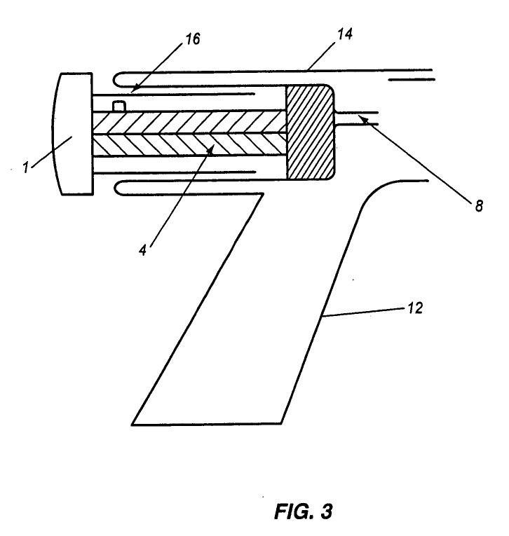

FIG. 3 is a schematic cross section of an alternative embodiment of the suction mechanism according to the present invention. In this embodiment, the position and mechanism of effecting the suction action has been slightly amended whereas handle 1 for puling the piston 4 has been allocated at the back of the device whereas it can be easily pulled out up to the lock point 16.

The device of the invention is working in a very easy and effective way with no need for any external suction device front part of the device is loaded with rubber bands 4-6. After directing the front part towards the vein which is to be legated, the user pull the handle 1 backwards and thereby activate a pulling force through the piston 4 and thereby pulling the said vain inside the device. Pressing button 6 will activate the lighting unit and the lamp will be switched on to enlighten the operation area. Meanwhile, pressing the handle 11 backward like triggering will release the rubber band to split around the base line of the vein. Releasing the triggering handle 11 will automatically insert a new rubber band ready for the second triggering.

I claim:

1. Device for ligation of veins of the hemorrhoid characterized by comprising:
a front part comprising a fixed pipe surrounded by a movable pipe;
a triggering cylinder with a triggering handle of elongated cylinder construction connected between the rear of a third part and the front part, and
said third part being a rear part or mechanical handle part comprising a cylinder 14 and handle 12 for holding the device.

2. Device for ligation of veins of the hemorrhoid according to claim 1 wherein the cylinder 14 has a top handle 1 which can be pulled back through a channel 2 up to a locking point 16.

3. Device for ligation of veins of the hemorrhoid according to claim 2 wherein in the front part of the cylinder 14 there is a lamp 7 that focus the light in the direction of the front part 10 to give the surgeon more concentrated lights on a working area.

4. Device for ligation of veins of the hemorrhoid of claim 3 wherein the handle 12 is used to hold the device and direct the front part towards the working area, the handle comprising a small button 6 which activates a lighting lamp on pressing.

5. Device for ligation of veins of the hemorrhoid of claim 1 wherein the handle of the pulling mechanism is located at the back of the device.

6. Device for ligation of veins of the hemorrhoid of claim 1 including 4-6 rubber bands.

7. Device for ligation of veins of the hemorrhoid of claim 1 including 3 rubber bands.

8. Device for ligation of veins of the hemorrhoid according to claim 1 characterized in that it has a built in mechanism for effecting suction of veins into the front part of the device.

9. Device for ligation of veins of the hemorrhoid according to claim 7 wherein the said mechanism includes a handle connected to a piston and sucking action is effected by pulling the handle with the connected piston backward.
